(19) United States
(12) Patent Application Publication
(10) Pub. No.: US 2019/0055527 A1
(43) Pub. Date: Feb. 21, 2019

(54) DNA POLYMERASE VARIANT

(71) Applicants: KYUSHU UNIVERSITY, NATIONAL UNIVERSITY CORPORATION, Fukuoka-shi, Fukuoka (JP); TAKARA BIO INC., Kusatsu-shi, Shiga (JP)

(72) Inventors: Yoshizumi ISHINO, Fukuoka-shi, Fukuoka (JP); Sonoko ISHINO, Fukuoka-shi, Fukuoka (JP); Takeshi YAMAGAMI, Fukuoka-shi, Fukuoka (JP); Takashi UEMORI, Otsu-shi, Shiga (JP); Nariaki TAKATSU, Kusatsu-shi, Shiga (JP)

(73) Assignees: KYUSHU UNIVERSITY, NATIONAL UNIVERSITY CORPORATION, Fukuoka-shi, Fukuoka (JP); TAKARA BIO INC., Kusatsu-shi, Shiga (JP)

(21) Appl. No.: 15/772,614

(22) PCT Filed: Nov. 24, 2016
(86) PCT No.: PCT/JP2016/084808

§ 371 Date: May 1, 2018

(30) Foreign Application Priority Data
Nov. 27, 2015 (JP) 2015-231974

(51) Int. Cl. C12N 9/12; C12P 19/34; C07K 14/195

(52) U.S. Cl. CPC C12N 9/1252; C12Y 207/07007; C07K 2319/00; C07K 14/195; C12P 19/34

(57) ABSTRACT

The present invention relates to a fusion polypeptide containing, in a direction of from an N-terminal side to a C-terminal side, one or more peptides which bind to a PCNA, and a polypeptide having a DNA polymerase activity; a method for amplifying nucleic acids using the polypeptide; and a composition and a kit, containing the polypeptide. According to the present invention, it is made possible to amplify a long-strand DNA in a short time in amplifying nucleic acids in the presence of PCNA even with a Pol I-type DNA polymerase.

### TECHNICAL FIELD

The present invention relates to a DNA polymerase variant. The DNA polymerase variant of the present invention is particularly useful in amplifying nucleic acids in the presence of PCNA.

### BACKGROUND ART

DNA polymerases are enzymes capable of freshly synthesizing a DNA strand in line with a DNA strand serving as a template in vitro, and a DNA strand is freshly synthesized so long as there are, besides a template DNA, an oligonucleotide that serves as primers and four kinds of deoxynucleotides (dATP, dGTP, dCTP, and dTP) in the reaction. The DNA polymerases have been utilized in numerous manipulations such as methods for amplifying nucleic acids including nucleotide sequencing and a polymerase chain reaction (PCR).

As the associated factors which improve DNA synthesis-related various properties (extensibility, speediness, accuracy, etc.) of DNA polymerases, various proteins have been found from thermophilic archaebacteria. As the associated factors, for example, plural proteins derived from Pyrococcus furiosus have been isolated (Patent Publication 1). In addition, as the associated factors, PCNA (proliferating cell nuclear antigen), RFC-S (replication factor C small subunit), or RFC-L (replication factor C large subunit) have been isolated from Thermococcus kodakarensis KOD1 strain (Patent Publication 2).

The PCNA, as a homopolymer, forms a cyclic structure called “sliding clamp,” which accelerates a DNA synthesis reaction. The PCNA is highly conserved from yeasts to human, and in eukaryotic cells a PCNA plays an important role in cell divisions, DNA replications, repairs, cell cycle regulations, or post-replication modifications such as DNA methylation and chromatin remodeling.

The RFC (replication factor C) is a protein complex composed of five subunits, and is also called “clamp loader” from its function of loading PCNA to DNA. Also, the RFC is equivalent to a γ-complex of Escherichia coli. The functions of the RFC as a clamp loader will be explained as follows: (1) An RFC binds to a DNA strand; (2) using energy generated by hydrolysis of ATP, an RFC opens a cyclic PCNA; (3) PCNA clamps a DNA strand; and (4) the ATP is further hydrolyzed, whereby the RFC is dissociated from the DNA, and the PCNA binds to the DNA.

A PCNA forms a complex with various proteins other than DNA polymerases and an RFC, and is involved in repairs and replications of a DNA and other genetic controlling functions. It has been known that in human at least twelve proteins bind to a PCNA. Each of the proteins binds to a PCNA via a PIP box (PCNA interaction protein box), so that the protein would be detained on a DNA strand.

It has been elucidated that there are some amino acid sequences highly homologous in the PIP box, and that some proteins bind to a PCNA via a PIP box site (Non-Patent Publication 1).

As mentioned above, a PCNA and an RFC cooperatively functions in nature to carry out DNA replications. Utilizing a PCNA which particularly plays a central role among them, an attempt has been made to improve the efficiency of PCR. A family A (Pol I-type) DNA polymerase derived from thermophilic eubacteria Thermus aquaticus (which is also referred to as “Taq polymerase”), which is widely used in PCR, does not interact with a PCNA. However, it has been reported that a chimeric fusion protein (chimeric Taq) in which 50 amino acids including a PIP box derived from Pol B of Archaeoglobus fulgidus are fused to a Taq polymerase at a C-terminal, so as to have the same form as a family B (α-type) DNA polymerase, amplifies the DNA in the presence of a PCNA derived from A. fulgidus. However, when the size of an amplified DNA was 5 kb, bands ascribed to the synthesized products became thin, so that there were still some disadvantages in extensibility (Non-Patent Publication 2).

### PRIOR ART PUBLICATIONS

### Patent Publications

Patent Publication 1: WO 1999/00506
Patent Publication 2: Japanese Patent Laid-Open No. 2002-360261

### Non-Patent Publications

Non-Patent Publication 1: Genes to Cells, 7(9), 911-922 (2002)
Non-Patent Publication 2: J. Biol. Chem., 277(18), 16179-16188 (2002)

### SUMMARY OF THE INVENTION

### Problems to be Solved by the Invention

As mentioned above, in the prior art, it can hardly be said that a DNA polymerase is provided which satisfies all of various properties such as extensibility, speediness, and accuracy.

The present invention is aimed at solving the problem of the conventional family A (Pol I-type) DNA polymerases as described above, and an object thereof is to provide a DNA polymerase which is more convenient and easy to use, and has excellent extensibility and speediness, and a method for amplifying nucleic acids using the polymerase.

### Means to Solve the Problems

The present inventors have intensively studied for the purpose of providing a novel family A (Pol I-type) DNA polymerase which can be utilized for amplifying nucleic acids in the presence of PCNA, and as a result, found that a reaction for amplifying nucleic acids is accelerated in the presence of a PCNA by utilizing a fusion polypeptide containing, in a direction of from an N-terminal side to a C-terminal side, one or more peptides which bind to a PCNA, and a polypeptide having a DNA polymerase activity. Thus, the present invention was completed.

Summarizing the present invention, the present invention relates to:

[1] a fusion polypeptide containing, in a direction of from an N-terminal side to a C-terminal side,
a) one or more peptides which bind to a PCNA, and
b) a polypeptide having a DNA polymerase activity;
[2] the fusion polypeptide according to [1], characterized in that the peptide which bind to a PCNA is a peptide comprising a PIP box;
[3] the fusion polypeptide according to [2], characterized in that the PIP box is a peptide consisting of any one of amino acid sequences shown in SEQ ID NOs: 52 to 91 of the Sequence Listing;
[4] the fusion polypeptide according to [1] or [2], characterized in that the peptide which bind to a PCNA is a peptide comprising a PIP box derived from a DNA polymerase-associated factor,
[5] the fusion polypeptide according to any one of [1], [2] and [4], characterized in that the peptide which bind to a PCNA is a peptide comprising a PIP box derived from a replication factor C large subunit;
[6] the fusion polypeptide according to any one of [1], [2], [4], and [5], characterized in that the peptide which bind to a PCNA is the amino acid sequence shown in SEQ ID NO: 3 of the Sequence Listing, or a peptide containing the above amino acid sequence;
[7] the fusion polypeptide according to any one of [1] to [6], characterized in that the fusion polypeptide comprises a 5 to 50 amino acid linker peptide between the peptide which bind to a PCNA and the polypeptide having a DNA polymerase activity;
[8] the fusion polypeptide according to [7], characterized in that the linker peptide is an amino acid sequence composed of serine and glycine;
[9] the fusion polypeptide according to any one of [1] to [8], characterized in that the polypeptide having a DNA polymerase activity is a Pol I-type DNA polymerase or a fragment thereof;
[10] the fusion polypeptide according to any one of [1] to [9], characterized in that the polypeptide having a DNA polymerase activity is a Taq DNA polymerase or a fragment thereof;
[11] a nucleic acid encoding a fusion polypeptide as defined in any one of [1] to [10];
[12] a composition for amplifying nucleic acids containing a fusion polypeptide as defined in any one of [1] to [10];
[13] the composition for amplifying nucleic acids according to [12], further containing a PCNA;
[14] a kit containing a fusion polypeptide as defined in any one of [1] to [10];
[15] the kit according to [14], further containing a PCNA; and
[16] a method for producing a DNA complementary to a template DNA, characterized by the use of a composition containing a fusion polypeptide as defined in any one of [1] to [10] and a PCNA.

### Effects of the Invention

According to the present invention, even a Pol I-type DNA polymerase, in the presence of PCNA, can amplify a long-strand DNA in a short time.

### BRIEF DESCRIPTION OF THE DRAWINGS

FIG. 1 is a photograph of SDS-PAGE gel relating to purification of a Taq DNA polymerase variant in Example 1. By analysis of SDS-PAGE, purities of Taq81 to Taq85 were confirmed.

FIG. 2 is a photograph of SDS-PAGE gel relating to purification of a Taq DNA polymerase variant in Example 1. By analysis of SDS-PAGE, purities of Taq92 to Taq94 were confirmed.

FIG. 3 is charts showing physical interaction analyses using a surface plasmon resonance (SPR) method in Example 2. By the SPR analyses, physical interactions between Taq DNA polymerase variants (Taq81 to Taq85 and Taq94) and PfuPCNA were measured.

FIG. 4 is a picture showing the results of amplifying a 1 kb DNA in Example 3(1). By PCR, DNA amplification abilities of Taq81 to Taq85 were confirmed.

FIG. 5 is a picture showing the results of amplifying an 8 kb DNA in Example 3(2). By PCR, DNA amplification abilities of Taq81 to Taq85 in the presence of PCNA were confirmed.

FIG. 6 is a picture showing the results of amplifying an 8 kb DNA in Example 3(2). By PCR, DNA amplification abilities of Taq92 to Taq94 in the presence of PCNA were confirmed.

FIG. 7 is a picture showing the results of amplifying a 12 kb DNA in Example 3(3). By PCR, DNA amplification abilities of Taq81 to Taq85 in the presence of PCNA were confirmed.

FIG. 8 is a picture showing the results of amplifying a 15 kb DNA in Example 3(3). By PCR, DNA amplification abilities of Taq81 to Taq85 in the presence of PCNA were confirmed.

FIG. 9 is a picture showing the results of amplifying a 12 kb DNA by PCR in Example 4. By PCR, DNA amplification abilities of Taq95 to Taq98 in the presence of PCNA were confirmed.

FIG. 10 is a graph showing relative values of amplifying a 12 kb DNA by PCR in Example 4.

### MODES FOR CARRYING OUT THE INVENTION

### Definitions, Etc.

The term “peptide” as used herein refers to a compound in which two or more amino acid molecules are bonded by removing one water molecule from an amino group of one amino acid molecule and a carboxyl group of the other amino acid molecule. In general, those composed of about 10 or less amino acids are referred to as oligopeptides, and those composed of equal to or greater than the above number of amino acids are referred to as polypeptides, but there are no strict boundaries therebetween.

The term “fusion polypeptide” as used herein refers to a polypeptide comprising two or more polypeptides which are not fused in a natural state, and a polypeptide comprising a peptide and a polypeptide that is not fused in a natural state.

The term “PCNA” as used herein is an abbreviation for proliferating cell nuclear antigen, and is a constituent of a protein molecule called “sliding clamp” from its unique shape and functions. The PCNA is a replication cofactor that forms a ring-shaped structure as a homopolymer, clamps a DNA strand in its central hole, and binds to a DNA polymerase at its surface to detain the enzyme on the DNA, thereby accelerating a DNA strand synthesis reaction. The PCNA is highly conserved from yeasts to human, and in eukaryotic cells, PCNA plays important roles in cell divisions, DNA replications, repairs, cell cycle regulations, or post-replication modifications such as DNA methylation and chromatin remodeling. In addition, in the present invention, all the proteins having the above functions are embraced within the PCNAs even though the names differ.

The phrase “polypeptide having a DNA polymerase activity” as used herein refers to a polypeptide having an activity of synthesizing a DNA strand complementary to a template nucleic acid (DNA or RNA) using deoxyribonucleotide triphosphate as a substrate. In the present invention, a known DNA polymerase or a variant thereof can be used as the “polypeptide having a DNA polymerase activity.”

As to the DNA polymerase in the present invention, when referred to “activity,” unless specified otherwise, the activity includes a DNA synthesizing activity and a primer extension activity. The DNA synthesizing activity includes an activity of using a DNA as a template, and synthesizing a DNA complementary thereto; and an activity of using an RNA as a template, and synthesizing a DNA complementary thereto. The DNA synthesizing activity can be measured as an uptake activity of a substrate deoxyribonucleotide triphosphate (dNTP) as well known to the person skilled in the art. More concretely, in a complementary strand synthesis reaction using a template such as a calf thymus DNA or a salmon sperm DNA, which is partially digested with DNaseI, and dNTP labeled with a radioactive isotope, the DNA polymerase activity is measured as an amount of the uptake of radioactive isomer into the complementary strand. This method is called a nucleotide uptake assay, and is also a standard method for measuring a DNA polymerase activity. Alternatively, the activity of a DNA polymerase can be evaluated by measuring a chain length of a primer extension product synthesized by a DNA polymerase using a template DNA hybridized with a primer as a substrate.

The present invention will be explained in detail hereinbelow.

(1) Fusion Polypeptide of the Present Invention

The fusion polypeptide of the present invention contains, in a direction of from an N-terminal side to a C-terminal side,

a) one or more peptides which bind to a PCNA, and
b) a polypeptide having a DNA polymerase activity.
Accordingly, the fusion polypeptide of the present invention can be said to be a DNA polymerase variant.

The phrase “peptides which bind to a PCNA” constituting the above fusion polypeptide is not particularly limited, so long as the peptides have the abilities of binding to a PCNA. Examples of the peptide include peptides containing a PIP box, which are the peptides existing in various PCNA-bindable proteins. The PIP box is an amino acid sequence existing in a protein interacting with a PCNA, and serves to detain the protein via the PCNA on the DNA strand. Here, in the present invention, all the peptides having the above functions would be embraced in the PIP boxes even though the names differ. For example, it has been known that thermophilic bacteria proteins involved in DNA replications or the like (for example, replication factor C large subunit etc.) have a PIP box. In the present invention, for example, examples of a preferred PIP box include, but not particularly limited to, an oligopeptide composed of at least eight amino acids, denoted by A1-A2-A3-A4-A5-A6-A7-A8, wherein A1 is glutamine residue, each of A2 and A3 is any amino acid residues, A4 is an amino acid residue selected from the group consisting of leucine residue, isoleucine residue, and methionine residue, each of A5 and A6 is any amino acid residues, A7 is phenylalanine residue or tryptophan residue, and A8 is an amino acid residue selected from the group consisting of phenylalanine residue, tryptophan residue, or leucine residue. Especially preferred includes one shown in SEQ ID NO: 3 of the Sequence Listing including eight amino acids QATLFDFL. Further, in the present invention, the peptide may be an oligopeptide containing 9 amino acids in which the above oligopeptide of eight amino acids further comprises lysine residue at an N-terminal side thereof. Examples of the amino acid sequences of the PIP box usable in the present invention are shown in Table 1 without intending to particularly limit the present invention thereto.

TABLE 1
[Table 1-1]

| | | Amino | SEQ ID NO. |
|---|---|---|---|
| Name of | | Acid | in Sequence |
| Protein | Organism Species | Sequence | Listing |
| RFC-L | P. furiosus | QATLFDFL | 52 |
| | M. jannaschii | QLTLDAFF | 53 |
| PolBI | P. furiosus | QVGLTSWL | 54 |
| | T. litoralis | QTGLDAWL | 55 |
| | A. fulgidus | QMSLDSFF | 56 |
| PolBII | P. occultum | QRSLFDFF | 57 |
| DP2 | M. jannaschii | QVKLSDFF | 58 |
| | M. thermoautotrophicum | QSSLDVFL | 59 |
| Polδ p66 | H. sapiens | QVSITGFF | 60 |
| Pol32 | S. cerevisiae | QGTLESFF | 61 |
| Cdc27 | S. pombe | QKSIMSFF | 62 |
| Pol2 | S. cerevisiae | QTSLTKFF | 63 |
| Fen1(RAD2) | P. furiosus | QSTLESWF | 64 |
| | M. jannaschii | QKTLDAWF | 65 |
| | A. fulgidus | QATLERWF | 66 |
| | H. sapiens | QGRLDDFF | 67 |
| | M. musculus | | |
| | X. laevis | | |
| | D. melanogaster | QVRLDSFF | 68 |
| | S. cerevisiae | QGRLDGFF | 69 |
| | S. pombe | QGRLDSFF | 70 |

[Table 1-2]

| | | Amino | SEQ ID NO. |
|---|---|---|---|
| Name of | | Acid | in Sequence |
| Protein | Organism Species | Sequence | Listing |
| DNA ligase I | H. sapiens | QRSIMSFF | 71 |
| | M. musculus | | |
| | X. laevis | QRTIKSFF | 72 |
| | S. cerevisiae | QATLARFF | 73 |
| | S. pombe | QSDISNFF | 74 |
| MSH3 | H. sapiens | QAVLSRFF | 75 |
| | S. cerevisiae | QPTISRFF | 76 |
| MSH6 | H. sapiens | QSTLYSFF | 77 |
| | S. cerevisiae | QSSLLSFF | 78 |
| UNG2 | H. sapiens | QKTLYSFF | 79 |
| | M. musculus | | |
| UNG | S. cerevisiae | QTTIEDFF | 80 |
| hMYH | H. sapiens | QQVLDNFF | 81 |
| XPG | H. sapiens | QLRIDSFF | 82 |
| | M. musculus | | |
| | X. laevis | | |
| | C. elegans | QMRLDRFF | 83 |
| | S. cerevisiae | QKRINEFF | 84 |
| | S. pombe | QSNLTQFF | 85 |
| Cac1 | S. cerevisiae | QSRIGNFF | 86 |
| hRECQ5 | H. sapiens | QNLIRHFF | 87 |
| Rrm3 | S. cerevisiae | QQTLSSFF | 88 |
| Cdc25C | H. sapiens | QEELFNFF | 89 |
| p15 | H. sapiens | QKGIGEFF | 90 |
| DNA-dependent | H. sapiens | QLIIRNFW | 91 |
| protein kinase | | | |

Further, the PIP box used in the present invention includes, but not particularly limited to, those derived from proteins produced by thermophilic bacteria. Examples include preferably a PIP box derived from a replication factor C large subunit of thermophilic bacteria, and more preferably a PIP box derived from a replication factor C large subunit of Pyrococcus furiosus. Alternatively, it may be a functional equivalent having substantially same level of activity as those mentioned above.

In addition, these PIP boxes may exist in plurality within a fusion polypeptide of the present invention. Examples of the number of PIP boxes contained in the fusion polypeptide include, but not particularly limited to from 1 to 6, and preferably from 2 to 4. These plural PIP boxes may each have amino acid sequences different from each other, so long as they play their roles. In addition, between the plural PIP boxes themselves, other amino acid sequences, for example, a linker peptide mentioned later may be inserted.

Further, a “linker peptide” may be present at a C-terminal side of the above PIP box. The term “linker peptide” constituting the fusion polypeptide of the present invention refers to a peptide which is inserted between polypeptides that are fused together or between a peptide and a polypeptide in the fusion polypeptide of the present invention in order to avoid the inhibition of their functions or folding. The length of the linker peptide includes, but not particularly limited to, peptides of from 3 to 100 amino acids, preferably 5 to 50 amino acids. The kinds of the amino acids constituting the linker peptide are not particularly limited, and it is better to avoid a linker which itself forms a complicated conformation, and a peptide with a relatively small side chain richly containing amino acids, for example, serine or glycine, is well used. It is preferable that the linker peptide in the present invention is amino acids composed of serine and glycine.

The “polypeptide having a DNA polymerase activity” is present at a C terminal of the above linker peptide. As the “polypeptide having a DNA polymerase activity” which constitutes the fusion polypeptide of the present invention, a known DNA polymerase or a variant thereof can be used. When the fusion polypeptide of the present invention is used in PCR, a thermostable DNA polymerase and a variant thereof, preferably a thermostable family A (Pol I-type) DNA polymerase and a variant thereof, and more preferably a DNA polymerase derived from bacteria of the genus Thermus or a variant thereof is used as a “polypeptide having a DNA polymerase activity.” According to the present invention, the performance of the Taq DNA polymerase can be dramatically improved, even though the present invention is not particularly limited thereby. In the present invention, “Taq polymerase” or “Taq DNA polymerase” refers to a Pol I-type DNA polymerase derived from Thermus aquaticus. The amino acid sequence of this DNA polymerase and the nucleotide sequence encoding the amino acid sequence are each shown as SEQ ID NOs: 1 and 2 which is a part of the present specification. Further, a Pol I-type DNA polymerase from Thermus thermophilus or Thermus flavus can be also used in the present invention. In the present specification, examples of the polypeptide having a DNA polymerase activity include a full-length polypeptide of a Pol I-type DNA polymerase or a fragment thereof, and preferably a full-length polypeptide of a Taq DNA polymerase or a fragment thereof. Here, the fragments of these polymerases may be a natural form or a variant form, so long as they have a DNA polymerase activity. In addition, the fragments of these polymerases may be fragments of polymerases not having a PIP box in a natural form, or may be fragments of polymerases having a PIP box in a natural form. Further, in a case of a fragment of a polymerase having a PIP box in a natural form, the PIP box may be removed.

Examples of one embodiment of the present invention are a fusion polypeptide containing a PIP box derived from a replication factor C large subunit from P. furiosus and Taq DNA polymerase. The amino acid sequences of the fusion polypeptides are shown in SEQ ID NOs: 4, 6, 8, 10, 12, 20, 22, 24, and 26.

It is preferable that the fusion polypeptide of the present invention has a dissociation constant (Kd) from a PCNA preferably within the range of from 1×10⁻⁸ to 25×10⁻⁷ M, preferably from 3×10⁻⁸ to 15×10⁻⁷ M, and more preferably from 5×10⁻⁸ to 10×10⁻⁷ M.

Further, the fusion polypeptide of the present invention can amplify a long-strand DNA, as compared to polypeptides having a DNA polymerase activity not having a peptide which binds to a PCNA. Especially, the fusion polypeptide can amplify a DNA having a length of 8 kb or more, preferably 12 kb or more, and more preferably 15 kb or more. Therefore, it can be said that the fusion polypeptide of the present invention is a DNA polymerase having excellent extensibility. Further, the fusion polypeptide of the present invention can amplify a DNA in a short time, as compared to a polypeptide having a DNA polymerase activity not having a peptide which binds to a PCNA. In other words, the fusion polypeptide of the present invention is a DNA polymerase having excellent speediness. Concretely, the fusion polypeptide of the present invention, as compared to a polypeptide before fusion, can shorten the time period that is required for DNA extension. For this reason, the fusion polypeptide is very useful in reactions for amplifying nucleic acids in which the DNA extension time is set shorter than a conventional method. Further, since the DNA extension time for each cycle can be shortened, it is possible to shorten the entire required time for the method for amplifying nucleic acids than a conventional method. For example, it is possible to amplify a DNA having a length of 8 kb by carrying out 30 cycles of shuttle PCR, wherein one cycle is 99° C. for 5 seconds and 66° C. for 4 minutes. It is possible to amplify a DNA having a length of preferably 12 kb or more, and more preferably 15 kb or more by carrying out 30 cycles of shuttle PCR, wherein one cycle is 99° C. for 5 seconds and 66° C. for 12 minutes.

(2) Nucleic Acid Encoding Fusion Polypeptide of the Present Invention

The present invention provides a nucleic acid encoding a fusion polypeptide as defined in the above (1). A nucleic acid of the present invention is incorporated into a recombinant vector, whereby a fusion polypeptide of the present invention can be produced in accordance with a method well known to the person skilled in the art. In the nucleotide sequence of the nucleic acid of the present invention, a nucleotide may be substituted so as to have an optimal codon in expression of the fusion polypeptide in the host cells. Next, a nucleic acid of the present invention is inserted downstream of a promoter of an appropriate expression vector to generate an expression vector. It is necessary to incorporate the above nucleic acid into a vector so that a fusion polypeptide of the present invention is expressed in a host, and the vector can contain, besides the promoter, a ribosome-binding sequence (e.g., SD sequence: Shine-Dalgarno sequence), a cis element such as a terminator or an enhancer, a selection marker (e.g., dihydrofolate reductase gene, ampicillin-resistant gene, neomycin-resistant gene) or the like. The transformant capable of producing a fusion polypeptide of the present invention can be obtained by transducing an expression vector mentioned above to a host cell.

Also, the nucleic acid of the present invention mentioned above may further contain a nucleic acid encoding an affinity tag in order to facilitate purification of a protein expressed. The nucleic acid encoding an affinity tag is, for example, a nucleic acid encoding histidine (His) tag, a glutathione S-transferase (GST) tag, a maltose binding protein (MBP) tag, a Strep(II) tag consisting of eight amino acid residues (Trp-Ser-His-Pro-Gln-Phe-Glu-Lys), and the like, without intending to limit the present invention thereto. The position at which the tag is added may be either one of a 5′-terminal side or a 3′-terminal side of a nucleic acid encoding a fusion polypeptide of the present invention, and the tag may be properly added at a position that would not be a hindrance to expression and tag functions. Here, it is preferable that the tag can be cleaved in the purification stage of the expressed protein.

As the expression vector, a vector capable of autonomous replication or a vector capable of being incorporated into a host chromosome can be used. As the vector, for example, a plasmid vector, a phage vector, a virus vector or the like can be used. As the plasmid vector, a plasmid which is suitable for a host to be used, for example, a plasmid derived from Escherichia coli, a plasmid derived from bacteria of the genus Bacillus, and a plasmid derived from yeasts are well known to the person skilled in the art, and many of them are commercially available. In the present invention, these known plasmids or modified forms thereof can be used. As the phage vector, for example, a λ phage (e.g., Charon4A, Charon21A, EMBL3, EMBL4, λgt10, λgt111, λZAP) or the like can be used, and as the virus vector, for example, an animal virus such as a retrovirus or a vaccinia virus, or an insect virus such as a baculovirus can be used.

As host cells, any one of prokaryotic cells, yeasts, animal cells, insect cells, plant cells, and the like can be used so long as the fusion polypeptide of the present invention can be expressed.

When a prokaryotic cell is used as a host cell, for example, bacteria belonging to the Escherichia genus such as Escherichia coli, bacteria belonging to the Bacillus genus such as Bacillus subtilis, bacteria belonging to the Pseudomonas genus such as Pseudomonas putida, bacteria belonging to the Rhizobium genus such as Rhizobium meliloti can be used as host cells. Escherichia coli which can be used in the production of heterologous proteins is well known to the person skilled in the art, and many of them are commercially available (e.g., Escherichia coli BL21, E. coli XL1-Blue, E. coli XL2-Blue, E. coli DH1, E. coli JM109, E. coli HB101, etc.). Also, Bacillus subtilis MI114, B. subtilis 207-21 or the like, which is a bacterium belonging to the Bacillus genus, or Brevibacillus choshinensis or the like, which is a bacterium belonging to the Brevibacillus genus, has been known as a host for production of heterologous protein. These host cells can be combined with an appropriate expression vector and used in the production of a fusion polypeptide of the present invention. In this case, a promoter which is carried on an expression vector can be selected depending upon a host, and, for example, in Escherichia coli, a promoter derived from Escherichia coli, a phage etc., such as a trp promoter, a lac promoter, a PL promoter, or a PR promoter, or a modified product thereof can be used, without intending to limit to those mentioned above. Further, an expression system (e.g., pET expression system, etc.) in which a promoter derived from a phage and an RNA polymerase gene are combined may be utilized. Further, a heterologous protein expression system in which an yeast, an insect cell or a mammalian cell is used as a host has been numerously constructed, and has already been commercially available. In the production of a fusion polypeptide of the present invention, these expression systems may be used.

The method for introducing an expression vector into a host is not particularly limited, so long as the method is capable of introducing a nucleic acid into a host, and, for example, a method using calcium ions, an electroporation method, a spheroplast method, a lithium acetate method or the like can be used. The method for introducing a recombinant vector into an insect cell is not particularly limited, so long as the method is capable of introducing a DNA into an insect cell, and, for example, a calcium phosphate method, a lipofection method, an electroporation method, or the like can be used. The infection of a phage vector or a virus vector to a host cell is carried out in accordance with a method depending on these vectors, whereby a transformant which expresses a fusion polypeptide of the present invention can be obtained.

A transformant into which an expression vector incorporated with a DNA encoding a fusion polypeptide of the present invention is transduced is cultured. The transformant can be cultured in accordance with an ordinary method usable in the cultivation of host cells. Depending upon the kinds of the promoters carried on an expression vector, appropriate induction procedures (addition of an inducer or modification of culture temperature) are carried out.

The fusion polypeptide of the present invention can be collected from a cultured product of the transformant. Here, the term “cultured product” includes all of culture supernatant, cultured cells, cultured bacteria, disruptions of cells or bacteria. When the fusion polypeptide of the present invention is accumulated in the cells of the transformant, a cultured product is centrifuged to harvest the cells, the cells are washed, and the cells are then disrupted to extract an intended protein, to provide a starting material for purification. When the fusion polypeptide of the present invention is secreted outside of the cells of the transformant, a cultured product is used directly, or culture supernatant obtained by removing the cells from a cultured product by centrifugation or the like is used as a starting material for purification. The fusion polypeptide of the present invention can be purified from the above starting material by solvent extraction, salting out with ammonium sulfate or the like, precipitation with an organic solvent, various chromatographies (ion exchange chromatography, hydrophobic chromatography, gel filtration, affinity chromatography or the like) or the like.

(3) Method for Amplifying Nucleic Acids Using Fusion Polypeptide of the Present Invention

The fusion polypeptide of the present invention can be used in amplifying long-strand length nucleic acids and amplifying nucleic acids with shortened reaction time period by combining the fusion polypeptide with a PCNA. The method for amplifying nucleic acids of the present invention can be used for any one of isothermal nucleic acid amplification method or temperature-changing nucleic acid amplification method. Any one of polymerase chain reaction (PCR), ligase chain reaction, MALBAC (Multiple Annealing and Looping-Based Amplification Cycles) method, MDA (Multiple Displacement Amplification) method, strand displacement DNA extension reaction (strand displacement amplification: SDA), rolling circle amplification (RCA) method, cross priming amplification method, loop-mediated isothermal amplification (LAMP) method, ICAN (isothermal and chimeric primer-initiated amplification of nucleic acids) method, or the like can be suitably used, without being particularly limited thereto. For example, a combination of the fusion polypeptide of the present invention with a PCNA can be expected to have high extensibility, so that it is effective in the preparation of a DNA having a long strand length for genome analysis or genome editing, and can be utilized in isothermal nucleic acid amplification method. Also, a combination of the fusion polypeptide of the present invention and a PCNA has excellent high-speed synthesis of DNA strand for improving PCR, so that it can be used for amplification of longer DNA with a shortened reaction time period. Although the present invention is not particularly limited thereto, a PCNA into which a mutation so as to lower stability of a ring-shaped structure is transduced is suitable to be combined with the fusion polypeptide of the present invention.

The PCNA used in the present invention includes a known PCNA or a variant thereof, and preferably a thermostable PCNA or a variant thereof is used. Examples are PCNA from P. furiosus or PCNA from T. kodakarensis, and the like, without particularly being limited thereto. Further, a variant PCNA can be also used in a composition for amplifying nucleic acids of the present invention. Examples of the variant PCNA are, for example, variant PCNAs described in International Publication Pamphlet WO 2007/004654, concretely variant PCNAs having a sequence in which an amino acid residue at 82nd, 84th, 109th, 139th, 143rd, or 147th position of PCNA from P. furiosus is substituted with another amino acid. An example of an especially preferred embodiment of the present invention is a variant PCNA having a sequence in which an amino acid residue at 143rd position is substituted from aspartic acid to asparagine (D143R). The variant PCNA of this embodiment exhibits especially excellent auxiliary actions with well-balanced extensibility and reaction speed of the DNA replication reaction, as shown in Examples set forth below.

Also, in the method for amplifying nucleic acids of the present invention, the fusion polypeptide of the present invention may be combined with a DNA polymerase that is different from that of the fusion polypeptide of the present invention. For example, a fusion polypeptide of the present invention which is generated using a Pol I-type DNA polymerase may be combined with an α-type DNA polymerase having 3′→5′ exonuclease activity and used in the method for amplifying nucleic acids. Here, a technique of performing PCR with a reaction solution containing two kinds of DNA polymerases having different 3′→5′ exonuclease activities has been known as LA-PCR (Long and Accurate PCR). Further, a combination of two kinds of fusion polypeptides of the present invention having different polypeptides having DNA polymerase activities may be used.

In addition, the oligonucleotide usable as a primer in the method for amplifying nucleic acids of the present invention has a sequence complementary to a nucleotide sequence of a nucleic acid used as a template, and the oligonucleotide is not particularly limited, so long as it hybridizes to a nucleic acid used as a template in the reaction conditions used. The strand length of the primer is preferably 6 nucleotides or more, and more preferably 10 nucleotides or more, from the viewpoint of specificity of hybridization, and the strand length is preferably 100 nucleotides or less, and more preferably 30 nucleotides or less, from the viewpoint of synthesis of the oligonucleotide. The above oligonucleotide can be chemically synthesized, for example, by a known method. In addition, the oligonucleotide may be an oligonucleotide derived from an organism sample, and, for example, an oligonucleotide may be prepared by isolating from a restriction endonuclease digest of a DNA prepared from a natural sample.

Furthermore, the method for amplifying nucleic acids of the present invention may be combined with a real-time detection technique. In the real-time detection, using an intercalator or a fluorescent-labeled probe, an amplified product is detected with the passage of time, concurrently with the amplification reaction. The intercalator includes SYBR(registered trademark) Green I and other nucleic acid-bindable pigments, and the fluorescent-labeled probe includes TaqMan(registered trademark) probe, CyCleave(registered trademark) probe, or molecular beacon probe, and the like, respectively.

(4) Composition for Amplifying Nucleic Acids Containing Fusion Polypeptide of the Present Invention

The fusion polypeptide obtained by the present invention can be used as a component for a composition for amplifying nucleic acids which can be used in the above (3) Method for Amplifying Nucleic Acids. Further, the composition for amplifying nucleic acids may contain elements essential for the activity of a DNA polymerase, for example, a divalent metal salt (magnesium salt, etc.), dNTP, buffering components for maintaining a pH, and the like.

Preferably, the composition for amplifying nucleic acids of the present invention can further contain a PCNA, in addition to the fusion polypeptide of the present invention. As the PCNA contained in the composition for amplifying nucleic acids, the PCNA explained in the above (3) or a variant thereof can be used.

Also, the composition of the present invention may contain a DNA polymerase which is different from that of the fusion polypeptide of the present invention. For example, the fusion polypeptide of the present invention generated by using a Pol I-type DNA polymerase may be combined with an α-type DNA polymerase having a 3′→5′ exonuclease activity to prepare a composition.

Examples of the divalent metal ions constituting the divalent metal salt contained in the composition of the present invention include magnesium ions, manganese ions, and cobalt ions. The divalent metal ions that are suitable for each of the DNA polymerases and concentrations thereof have been known in the art. The divalent metal ions can be supplied in the form of salts such as chlorides, sulfates, or acetates. Examples of the divalent metal ion concentration in the composition of the present invention are, for example, from 0.5 to 20 mM without particularly limiting the present invention thereto.

In the present invention, at least one member of dNTP, namely deoxyribonucleotide triphosphate (e.g., dATP, dCTP, dGTP, and dTTP) and derivatives thereof is used. Examples of the deoxyribonucleotide triphosphate contained in the composition of the present invention are preferably a mixture of four kinds dATP, dCTP, dGTP, and dTTP.

Also, the composition of the present invention may contain a buffering component. The component refers to, for example, a compound or a mixture having an action of moderating the fluctuations of a hydrogen ion concentration (pH) of a reaction solution, without particularly being limited thereto. In general, a mixed solution of a weak acid and a salt thereof or a weak base and a salt thereof has a strong buffering action, and is widely used for the purpose of a pH control as a reaction buffering agent. The pH of the composition of the present invention is appropriately set within an ordinary range for performing PCR, for example, within a pH range of from 8.0 to 9.5, without particularly limiting the present invention thereto.

Further, the composition of the present invention may contain a component for real-time detection. The composition can be combined with an intercalator or a fluorescent-labeled probe, without particularly being limited thereto.

(5) Kit Containing Fusion Polypeptide of the Present Invention

The kit containing a fusion polypeptide of the present invention is one embodiment of the present invention. Preferably, examples of the kit include a kit further containing a PCNA, in addition to a fusion polypeptide described in the above (1). In particular, the preferable PCNA contained in the kit includes the above-described variant PCNA in which a ring-shaped structure is made labile. The kit of the present invention may further contain a component usable in the preparation of a composition for amplifying nucleic acids of the present invention, such as a divalent metal salt (magnesium salts, etc.), dNTP, or a buffering component for maintaining a pH, as an individual component, or the kit may contain a component in which plural of these components are combined and prepared.

Further, a component for real-time detection may be contained as a component. Examples include an intercalator, a fluorescent-labeled probe, and the like, without particularly being limited thereto.

(6) Method for Producing DNA Complementary to Template DNA of the Present Invention

A composition containing a fusion polypeptide of the present invention and a PCNA can be used in a method for producing a DNA complementary to a template DNA. In the method for producing a DNA, the above (3) Method for Amplifying Nucleic Acids can be utilized. By utilizing a DNA produced by these nucleic acid amplification methods, it is possible to determine a nucleotide sequence of a target nucleic acid, to label a target nucleic acid, and to introduce a site-directed mutation to a target nucleic acid.

### EXAMPLES

The present invention will be specifically described hereinbelow by the Examples, without intending to limit the scope of the present invention thereto.

### Example 1 Purification of Taq DNA Polymerase to which PIP Box was Added

To an N-terminal or a C-terminal of a Taq DNA polymerase having the amino acid sequence shown in SEQ ID NO: 1 of the Sequence Listing was added a PIP box of replication factor C large subunit from Pyrococcus furiosis (hereinafter simply PfuRFCL) having the amino acid sequence shown in SEQ ID NO: 3 of the Sequence Listing via a linker peptide. The Taq DNA polymerases to which a PIP box was added to an N-terminal were five kinds of Taq81 to Taq85 (PIP-L5-Taq, PIP-L10-Taq, PIP-L15-Taq, PIP-L35-Taq, and PIP-L47-Taq), and the Taq DNA polymerases to which a PIP box was added to a C-terminal were three kinds of Taq92 to Taq94 (Taq-L5-PIP, Taq-L10-PIP, and Taq-L15-PIP). Further, a Taq DNA polymerase to which two to five PIP boxes were added to an N-terminal was prepared. Concretely, they were four kinds of Taq95 (PIP-L14-PIP-L15-Taq), Taq96 (PIP-L14-PIP-L14-PIP-L15-Taq), Taq97 (PIP-L14-PIP-L14-PIP-L14-PIP-L15-Taq), and Taq98 (PIP-L14-PIP-L14-PIP-L14-PIP-L14-PIP-L15-Taq). The names and structures of the Taq DNA polymerases to which PIP box or boxes were added in the present specification are shown in Table 2. The number following “L” in the column of “Structure” in Table 2 shows the length of a linker peptide (number of amino acid residues) composed of repeats of serine residues and glycine residues.

TABLE 2

| | | Amino Acid | Nucleotide |
|---|---|---|---|
| Name | Structure | Sequence | Sequence |
| Taq81 | PIP-L5-Taq | SEQ ID NO: 4 | SEQ ID NO: 5 |
| Taq82 | PIP-L10-Taq | SEQ ID NO: 6 | SEQ ID NO: 7 |
| Taq83 | PIP-L15-Taq | SEQ ID NO: 8 | SEQ ID NO: 9 |
| Taq84 | PIP-L35-Taq | SEQ ID NO: 10 | SEQ ID NO: 11 |
| Taq85 | PIP-L47-Taq | SEQ ID NO: 12 | SEQ ID NO: 13 |
| Taq92 | Taq-L5-PIP | SEQ ID NO: 14 | SEQ ID NO: 15 |
| Taq93 | Taq-L10-PIP | SEQ ID NO: 16 | SEQ ID NO: 17 |
| Taq94 | Taq-L15-PIP | SEQ ID NO: 18 | SEQ ID NO: 19 |
| Taq95 | PIP-L14-PIP-L15-Taq | SEQ ID NO: 20 | SEQ ID NO: 21 |
| Taq96 | PIP-L14-PIP-L14-PIP- | SEQ ID NO: 22 | SEQ ID NO: 23 |
| | L15-Taq | | |
| Taq97 | PIP-L14-PIP-L14-PIP- | SEQ ID NO: 24 | SEQ ID NO: 25 |
| | L14-PIP-L15-Taq | | |
| Taq98 | PIP-L14-PIP-L14-PIP- | SEQ ID NO: 26 | SEQ ID NO: 27 |
| | L14-PIP-L14-PIP-L15- | | |
| | Taq | | |

(1) Taq81 (PIP-L5-Taq) Expression Plasmid

Using a Taq DNA polymerase gene having the nucleotide sequence shown in SEQ ID NO: 2 of the Sequence Listing as a template, PCR was carried out with a TaqNPIP-5 primer having the nucleotide sequence shown in SEQ ID NO: 28 of the Sequence Listing and a Taq-3 primer having the nucleotide sequence shown in SEQ ID NO: 29 of the Sequence Listing.

As the enzyme for PCR, KOD Plus Neo DNA polymerase (manufactured by TOYOBO CO, LTD.) was used, and the conditions for PCR were 30 cycles of reaction, wherein one cycle is 95° C. for 30 seconds, 55° C. for 30 seconds, and 72° C. for 3 minutes. An amplified fragment was purified by agarose gel electrophoresis, and thereafter cleaved with restriction enzymes NdeI (manufactured by TAKARA BIO INC.) and NotI (manufactured by TAKARA BIO INC.). This fragment was ligated with pET21a (manufactured by Novagen) which was cleaved with the same restriction enzymes. Escherichia coli JM109 strain (manufactured by TAKARA BIO INC.) was transformed with the ligation product, and spread on an LB-ampicillin plate. A plasmid was purified from the colonies formed, nucleotide sequences were read off, and it was confirmed that the nucleotide sequence of SEQ ID NO: 5 was contained. This plasmid was named pTaq81.

(2) Taq82 (PIP-L10-Taq) Expression Plasmid

A DNA (SEQ ID NO: 7) encoding a polypeptide in which five amino acids were inserted between PIP box and a linker peptide of Taq81 was generated by a site-directed mutagenesis using a QuickChange site-directed mutagenesis kit (manufactured by Agilent Technologies).

Using pTaq81S as a template and primers of taqN10-5 and taqN10-3 having the nucleotide sequences shown in SEQ ID NOs: 30 and 31 of the Sequence Listing, respectively, 14 cycles of PCR were carried out, wherein one cycle is 95° C. for 30 seconds, 55° C. for 60 seconds, and 68° C. for 8 minutes. Subsequently, Escherichia coli JM109 strain was transformed with 1 μL of a reaction solution digested with DpnI, and the transformed cells were spread on an LB-ampicillin plate. A plasmid was purified from the colonies formed, nucleotide sequences were read off, and it was confirmed that the nucleotide sequence of SEQ ID NO: 7 was contained. This plasmid was named pTaq82.

(3) Taq83 (PIP-L15-Taq) Expression Plasmid

Taq83 (PIP-L15-Taq) expression plasmid in which a sequence corresponding to 10 amino acids was inserted between PIP box and a linker peptide of Taq81 was constructed according to the mutagenesis method described in Example 1(2).

In this mutagenesis, PCR was carried out with pTaq81 as a template, and changing the used primers to taqN15-5 and taqN15-3 having the nucleotide sequences shown in SEQ ID NOs: 32 and 33 of the Sequence Listing, respectively, and a plasmid containing the nucleotide sequence of SEQ ID NO: 9 was obtained. This plasmid was named pTaq83.

(4) Taq84 (PIP-L35-Taq) Expression Plasmid

A Taq84 (PIP-L35-Taq) expression plasmid in which a sequence corresponding to 20 amino acids was inserted between PIP box and a linker peptide of Taq83 was constructed according to the mutagenesis method described in Example 1(2).

In this mutagenesis, PCR was carried out with pTaq83 as a template, and two primers taq-plus20-5 and taq-plus20-3 having the nucleotide sequences shown in SEQ ID NOs: 34 and 35 of the Sequence Listing, respectively, and a plasmid containing the nucleotide sequence of SEQ ID NO: 11 was obtained. This plasmid was named pTaq84.

(5) Taq85 (PIP-L47-Taq) Expression Plasmid

A Taq85 (PIP-L47-Taq) expression plasmid in which a sequence corresponding to 12 amino acids was inserted between 14th and 15th amino acids of a linker peptide of Taq84 was constructed according to the mutagenesis method described in Example 1(2).

In this mutagenesis, PCR was carried out with pTaq84 as a template, and two primers of Taq-plus12-5 and Taq-plus12-3 having the nucleotide sequences shown in SEQ ID NOs: 36 and 37 of the Sequence Listing, respectively, and a plasmid containing the nucleotide sequence of SEQ ID NO: 13 was obtained. This plasmid was named pTaq85.

(6) Taq92 (Taq-L5-PIP) Expression Plasmid

Using a Taq DNA polymerase gene having the nucleotide sequence shown in SEQ ID NO: 2 of the Sequence Listing as a template, and two primers Taq-5 and Tq-L5-PIP-3 having the nucleotide sequences shown in SEQ ID NOs: 38 and 39 of the Sequence Listing, respectively, PCR, ligation, transformation, and plasmid purification were carried out according to the procedures described in Example 1(1). The plasmid thus obtained containing the nucleotide sequence of SEQ ID NO: 15 was named pTaq92.

(7) Taq93 (Taq-L10-PIP) Expression Plasmid

Using a Taq DNA polymerase gene having the nucleotide sequence shown in SEQ ID NO: 2 of the Sequence Listing as a template, and two primers Taq-5 and Tq-L10-PIP-3 having the nucleotide sequences shown in SEQ ID NOs: 38 and 40 of the Sequence Listing, respectively, PCR, ligation, transformation, and plasmid purification were carried out under the conditions described in Example 1(1). The plasmid thus obtained containing the nucleotide sequence of SEQ ID NO: 17 was named pTaq93.

(8) Taq94 (Taq-L15-PIP) Expression Plasmid

Using a Taq DNA polymerase gene having the nucleotide sequence shown in SEQ ID NO: 2 of the Sequence Listing as a template, and two primers Taq-5 and Tq-L15-PIP-3 having the nucleotide sequences shown in SEQ ID NOs: 38 and 41 of the Sequence Listing, respectively, PCR, ligation, transformation, and plasmid purification were carried out under the conditions described in Example 1(1). The plasmid thus obtained containing the nucleotide sequence of SEQ ID NO: 19 was named pTaq94.

(9) Taq95 (PIP-L14-PIP-L15-Taq) Expression Plasmid

A Taq95 (PIP-L14-PIP-L15-Taq) expression plasmid in which a sequence corresponding to PIP boxes and the linker peptides was inserted between PIP box and a linker peptide of Taq83 was constructed according to the mutagenesis method described in Example 1(2).

In this mutagenesis, PCR was carried out with pTaq83 as a template, and two primers Taq95-5 and Taq-3 having the nucleotide sequences shown in SEQ ID NOs: 48 and 29 of the Sequence Listing, respectively, and a plasmid containing the nucleotide sequence of SEQ ID NO: 21 was obtained. This plasmid was named pTaq95.

(10) Taq96 (PIP-L14-PIP-L14-PIP-L15-Taq) Expression Plasmid

A Taq96 (PIP-L14-PIP-L14-PIP-L15-Taq) expression plasmid in which a sequence corresponding to PIP boxes and the linker peptides was inserted between PIP box and a linker peptide of Taq95 was constructed according to the mutagenesis method described in Example 1(2).

In this mutagenesis, PCR was carried out with pTaq95 as a template, and two primers Taq96-5 and Taq-3 having the nucleotide sequences shown in SEQ ID NOs: 49 and 29 of the Sequence Listing, respectively, and a plasmid containing the nucleotide sequence of SEQ ID NO: 23 was obtained. This plasmid was named pTaq96.

(11) Taq97 (PIP-L14-PIP-L14-PIP-L14-PIP-L15-Taq) Expression Plasmid

A Taq97 (PIP-L14-PIP-L14-PIP-L14-PIP-L15-Taq) expression plasmid in which a sequence corresponding to PIP boxes and the linker peptides was inserted between PIP box and a linker peptide of Taq96 was constructed according to the mutagenesis method described in Example 1(2).

In this mutagenesis, PCR was carried out with pTaq96 as a template, and two primers Taq97-5 and Taq-3 having the nucleotide sequences shown in SEQ ID NOs: 50 and 29 of the Sequence Listing, respectively, and a plasmid containing the nucleotide sequence of SEQ ID NO: 25 was obtained. This plasmid was named pTaq97.

(12) Taq98 (PIP-L14-PIP-L14-PIP-L14-PIP-L14-PIP-L15-Taq) Expression Plasmid

A Taq98 (PIP-L14-PIP-L14-PIP-L14-PIP-L14-PIP-L15-Taq) expression plasmid in which a sequence corresponding to PIP boxes and the linker peptides was inserted between PIP box and a linker peptide of Taq97 was constructed according to the mutagenesis method described in Example 1(2).

In this mutagenesis, PCR was carried out with pTaq97 as a template, and two primers Taq98-5 and Taq-3 having the nucleotide sequences shown in SEQ ID NOs: 51 and 29 of the Sequence Listing, respectively, and a plasmid containing the nucleotide sequence of SEQ ID NO: 27 was obtained. This plasmid was named pTaq98.

(13) Preparation of Taq DNA Polymerase to which PIP Box was Added

Escherichia coli BL21-CodonPlus (DE3)-RIL (manufactured by Agilent Technologies) was transformed with each of the expression plasmids pTaq81 to pTaq85 and pTaq92 to pTaq94, and the transformant obtained was subjected to shaking culture in 1 L of an LB medium containing 50 μg/mL ampicillin and 34 μg/mL chloramphenicol. At a point where OD600 reached 0.2 to 0.3, IPTG was added so as to have a final concentration of 1 mM, and expression of a Taq DNA polymerase was induced. Thereafter, the reaction solution was further subjected to a shaking culture at 25° C. for about 18 hours. After culture, the cultured bacteria were harvested, and the cultured bacteria were washed with a PBS solution (150 mM NaCl, 20 mM Na₂HPO₄, 2 mM NaH₂PO₄, at pH 7.5). Thereafter, the cultured bacteria were again harvested and stored at −80° C.

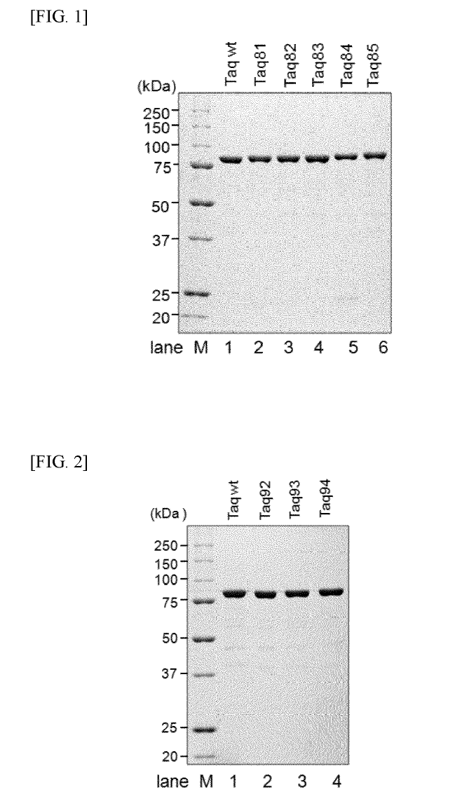

To the frozen cultured bacteria was added 20 mL of a solution A (50 mM Tris-HCl, 1 mM EDTA, pH 8.0) containing 1 mM PMSF (manufactured by nacalai tesque), and the mixture was subjected to ultrasonic disruption (on for 10 seconds, off for 10 seconds/on for a total of 10 minutes) on ice. The lysate was centrifuged (23,708×g) at 4° C. for 10 minutes, and the supernatant obtained was heat-treated at 75° C. for 30 minutes, and the heat-treated mixture was again centrifuged (23,708×g) at 4° C. for 10 minutes. NaCl was added to the supernatant obtained so as to have a final concentration of 1 M, and further a 5% (w/v) polyethyleneimine solution (pH 8.0) was added thereto so as to have a final concentration of 0.15%, and the mixture was allowed to stand on ice for 20 minutes. Thereafter, the mixture was centrifuged (23,708×g) at 4° C. for 10 minutes. Ammonium sulfate was gradually added to the supernatant obtained at a low temperature so as to be 80% saturation, the mixture was allowed to stand overnight at 4° C., and centrifuged (23,708×g) at 4° C. for 10 minutes. The precipitates obtained were suspended in a solution B (50 mM Tris-HCl, 10% glycerol, pH 8.0) containing 1 M ammonium sulfate, and the suspension was subjected to a hydrophobic column HiTrap Phenyl HP 5 mL (manufactured by GE Healthcare) using AKTA Explorer (manufactured by GE Healthcare) to elute the protein by a 1 M to 0 M ammonium sulfate concentration gradient using the solution B. The eluted fraction obtained was dialyzed against a solution C (50 mM Tris-HCl, 50 mM NaCl, pH 8.0) overnight, the dialyzed solution was then subjected to affinity column HiTrap Heparin HP 5 mL (manufactured by GE Healthcare) to elute the protein by a 50 mM to 1 M sodium chloride concentration gradient, and this eluted fraction was used as a final purification product. The final purification product was subjected to 10% SDS-PAGE, and detected by CBB staining. The results are shown in FIGS. 1 and 2.

As shown in FIGS. 1 and 2, Taq DNA polymerase variants (Taq81 to Taq85 and Taq92 to Taq94), fusion proteins in which the wild-type Taq DNA polymerase and PIP box were fused, could be purified in a single band. Also, Taq95 to Taq98 could be purified in a single band in the same manner.

### Example 2 Physical Interaction Analysis Using SPR Method

### (1) Method

In the surface plasmon resonance (SPR) analysis, BIAcore J (manufactured by BIACORE) was used. A PCNA from Pyrococcus furiosus (J. Bacteriology, 181, 6591-6599, 1999: hereinafter referred to as PfuPCNA) was immobilized on a CM5 sensor chip (manufactured by GE Healthcare) by amine coupling, and the measurement was carried out under the conditions of 25° C., a flow rate of 30 μL/minutes, and a solution E (10 mM Tris-HCl, 50 mM KCl, 1.5 mM MgCl₂, pH 8.3). A sample solution of Taq81 to Taq85 diluted to 10, 20, 40, 80, 160, or 320 nM or a sample solution of Taq94 diluted to 125 nM, 250 nM, 500 nM, 1 μM, or 2 μM was each added for 2 minutes. The sensorgrams obtained were analyzed by BIA evaluation program, and the dissociation constants (Kd) between Taq81 to Taq85 and PfuPCNA and between Taq94 and PfuPCNA were calculated.

### (2) Results

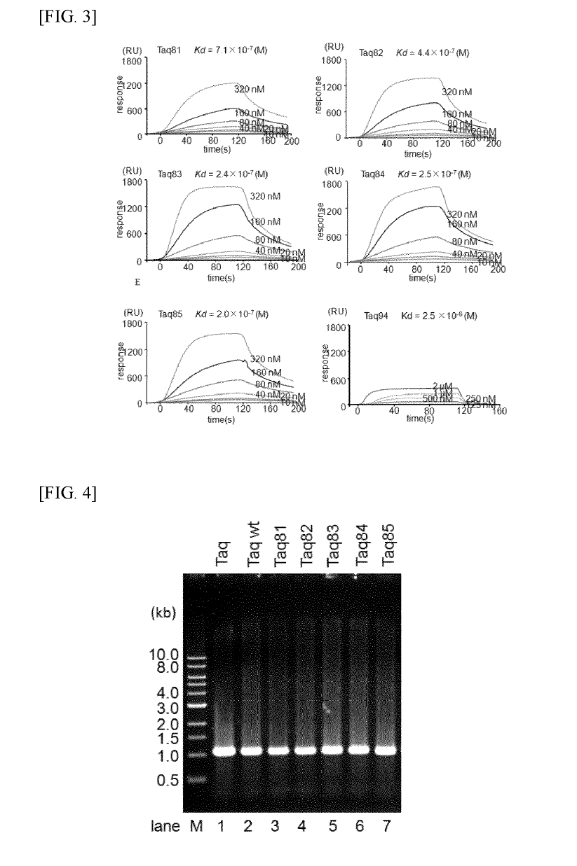

The results for the SPR analysis are shown in FIG. 3. The dissociation constants (Kd) with respect to the bindings between five kinds of Taq DNA polymerases to which PIP box was added at an N-terminal, each having a different length of a linker peptide (Taq81, Taq82, Taq83, Taq84, and Taq85), and PfuPCNA were calculated. As a result, the values were 7.1×10⁻⁷ M, 4.4×10⁻⁷ M, 2.4×10⁻⁷ M, 2.5×10⁻⁷ M, and 2.0×10⁻⁷ M, which were nearly of the same level regardless of the lengths of the linker peptides. The above results show that the strength of the interactions between the Taq DNA polymerase to which PIP box was added at an N-terminal and the PfuPCNA was nearly the same regardless of the length of the linker peptides.

On the other hand, the dissociation constant (Kd) between Taq94 and PfuPCNA was calculated to be 2.5×10⁻⁶ M. This is about 10 times of the dissociation constants of Taq81 to Taq85, and it could be confirmed from the results that the interaction was weak between the Taq DNA polymerase to which PIP box was added at a C-terminal and the PfuPCNA.

### Example 3 DNA Amplification by PCR

(1) Amplification of 1 kb DNA

Using Taq81 to Taq85 prepared in Example 1 (13), PCR was carried out with a lambda DNA as a template. The reaction solution composition was 1 nM Taq81 to Taq85, 10 mM Tris-HCl (pH 8.3), 50 mM KCl, 1.5 mM MgCl₂, 0.2 mM dNTP, and 1 ng lambda DNA, and 0.4 μM of each primer, and a final volume of the reaction solution was 50 μL. The reaction was carried out in 30 cycles, wherein one cycle is 95° C. for 30 seconds, 55° C. for 30 seconds, and 72° C. for 60 seconds.

In this PCR, two primers F1 and R2-2 having the nucleotide sequences shown in SEQ ID NOs: 42 and 43 of the Sequence Listing, respectively, were used. A product obtained was separated by 1% agarose gel, and stained with ethidium bromide. As a result, as shown in FIG. 4, all of a commercially available Taq DNA polymerase (lane 1), a wild-type Taq DNA polymerase (lane 2), and Taq81 to Taq85 (lanes 3 to 7) showed a band at a position of 1 kb. From the above results, it could be confirmed that the PIP box and the linker peptide added at an N-terminal do not affect PCR.

(2) Amplification of 8 kb DNA

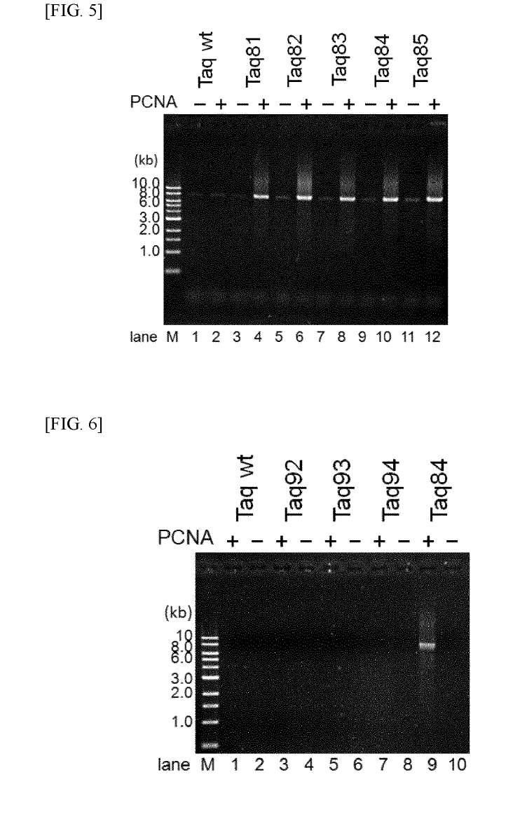

Next, PCR was carried out in the absence and in the presence of a PCNA. The reaction solution composition was 1 nM wild-type Taq DNA polymerase or Taq81 to Taq85, or 40 nM PufPCNA D143R variant (PCNA13) prepared by a method described in Examples of International Publication No. WO 2007/004654, 10 mM Tris-HCl (pH 8.3), 50 mM KCl, 1.5 mM MgCl₂, 0.2 mM dNTP, 1 ng of lambda DNA, 0.4 μM of each primer, and a final volume of the reaction solution was 50 μL. First, a reaction solution was incubated at 95° C. for 1 minute, and thereafter 30 cycles of shuttle PCR were carried out, wherein one cycle is 99° C. for 5 seconds, and 66° C. for 4 minutes. In this PCR, two primers LF-35 and LR8-35 having the nucleotide sequences shown in SEQ ID NOs: 44 and 45 of the Sequence Listing, respectively, were used. A product obtained was separated by 1% agarose gel, and stained with ethidium bromide. As a result, as shown in FIG. 5, contrary to the wild-type Taq DNA polymerases where hardly any bands were found at a position of 8 kb in either in the absence of a PCNA (lane 1) or in the presence of a PCNA (lane 2), Taq81 to Taq85 showed strong bands at a position of 8 kb in the presence of a PCNA (lanes 4, 6, 8, 10, and 12), although hardly any bands were found in the absence of the PCNA (lanes 3, 5, 7, 9, and 11).

The above results show that the Taq81 to Taq85, which are Taq DNA polymerase to which a PIP box is previously added at an N-terminal, are capable of amplifying an 8 kb DNA in the presence of a PCNA.

On the other hand, as shown in FIG. 6, Taq92 to Taq93, Taq DNA polymerases to which PIP box was added at a C-terminal, were not capable of amplifying an 8 kb DNA even in the presence of a PCNA.

(3) Amplification of 12 kb and 15 kb DNAs

The amplifications of 12 kb and 15 kb DNAs were studied. A reaction solution was prepared in the same manner as in Example 3-(2), except that in the amplification of 12 kb, two primers LF-35 and LR12-35 having the nucleotide sequences shown in SEQ ID NOs: 44 and 46 of the Sequence Listing, respectively, were used, or that in the amplification of 15 kb DNA, two primers LF-35 and LR15-35 having the nucleotide sequences shown in SEQ ID NOs: 44 and 47 of the Sequence Listing, respectively, were used.

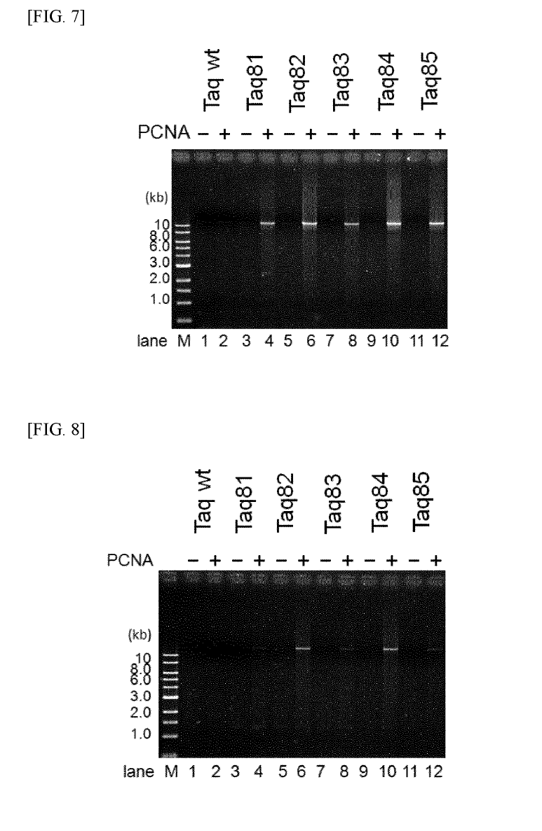

The conditions for PCR were as follows. First, a reaction solution was incubated for 95° C. for 1 minute, and subsequently, 30 cycles of shuttle PCR were carried out, wherein one cycle is 99° C. for 5 seconds, and 66° C. for 12 minutes. The analyses of a product obtained were carried out in the same manner as in Example 3(2). The results are shown in FIGS. 7 and 8.

As shown in FIG. 7, in the amplification of a 12 kb DNA, although the wild-type Taq DNA polymerase showed no bands in the absence of a PCNA (lane 1) and in the presence of a PCNA (lane 2), Taq81 to Taq85 (lanes 4, 6, 8, 10, and 12) showed a band appearing at a position of 12 kb in the presence of a PCNA. Likewise, as shown in FIG. 8, even in the amplification of a 15 kb DNA, bands could be confirmed in the presence of a PCNA in Taq81 to Taq85 (lanes 4, 6, 8, 10, and 12).

The above results show that Taq81 to Taq85 are capable of amplifying 12 kb and 15 kb DNAs in the presence of a PCNA.

In addition, the conditions for shuttle PCR employed in this Example are shorter in the time period needed for annealing of the primers and extension of complementary DNA, as compared to the conditions of conventional shuttle PCR. Nonetheless, it could be confirmed that a DNA of the same long-strand length as the conventional ones could be amplified, so that the shuttle PCR is excellent in speediness.

### Example 4 Taq DNA Polymerases to which Plural PIP Boxes were Added

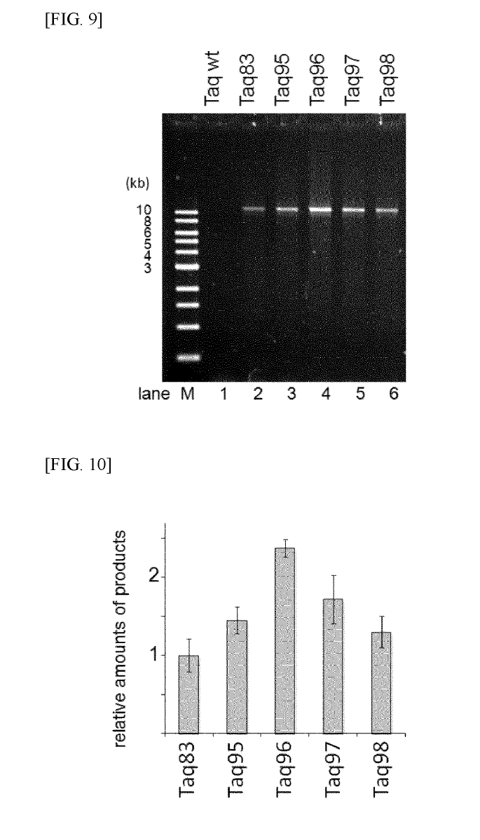

Using Taq95 (PIP-L14-PIP-L15-Taq), Taq96 (PIP-L14-PIP-L14-PIP-L15-Taq), Taq97 (PIP-L14-PIP-L14-PIP-L14-PIP-L15-Taq), and Taq98 (PIP-L14-PIP-L14-PIP-L14-PIP-L14-PIP-L15-Taq), each of which was prepared in Example 1(13), PCR was carried out for a 12 kb in the presence of a PCNA in the same manner as in Example 3(3). The results are shown in FIG. 9. Further, an amplified product was quantified using LAS-3000mini (manufactured by GE Healthcare). The procedures from PCR to quantification mentioned above were repeated 3 times, and a relative value was graphically shown, in which an amplified product of Taq83 (PIP-L15-Taq) was defined as 1. The results are shown in FIG. 10.

As shown in FIGS. 9 and 10, Taq95 to Taq98 to which plural PIP boxes were added could be confirmed to have larger amplified products as compared to Taq83 where PIP box was one. Especially, in Taq96 to which three PIP boxes were added, the amplified products were largest.

It is shown from the above that efficient DNA amplification can be made by adding plural PIP boxes to an N-terminal of the Taq DNA polymerase.

### Example 5 Taq DNA Polymerases to which PIP Box Comprising Various Sequences are Added

Using any one of peptides comprising amino acid sequences shown in SEQ ID NOs: 52 to 91 of the Sequence Listing listed in Table 1 (i.e., PIP box), in place of a PIP box comprising the amino acid sequence shown in SEQ ID NO: 3 of the Sequence Listing used in Example 1, Taq DNA polymerases to which any one of PIP boxes is added at an N-terminal are purified in accordance with the method described in Example 1. Using the Taq DNA polymerase, the amplification of a DNA is carried out in the presence of a PCNA in accordance with the method described in Example 3.

When any one of peptides listed in Table 1 are used in place of the PIP box comprising the amino acid sequence shown in SEQ ID NO: 3, the same effects as described above can be expected.

Further, PCR is carried out using a Taq DNA polymerase to which any one of peptides comprising amino acid sequences shown in SEQ ID NOs: 52 to 91 of the Sequence Listing listed in Table 1 (i.e., PIP box) are added at an N-terminal in plurality, in place of a PIP box comprising the amino acid sequence shown in SEQ ID NO: 3 of the Sequence Listing used in Example 1, in the presence of a PCNA, in accordance with the method described in Example 4.

When any one of peptides listed in Table 1 are used in place of the PIP box comprising the amino acid sequence shown in SEQ ID NO: 3, the same effects as described above can be expected.

Here, concrete primers to be used in the purification of a Taq DNA polymerase to which any of peptides comprising amino acid sequences shown in SEQ ID NOs: 52 to 91 are added, or concrete primers to be used in DNA amplification using the Taq DNA polymerase will be prepared referring to the description of the specification of the present application and the above Examples.

### INDUSTRIAL APPLICABILITY

According to the present invention, a family A (Pol I-type) DNA polymerase variant which is more convenient and easy-to-use, and has excellent extensibility and speediness, and a method for amplifying nucleic acids using the polymerase are provided. The DNA polymerase variant of the present invention is useful in amplifying nucleic acids especially in the presence of a PCNA.

### SEQUENCE LISTING FREE TEXT

SEQ ID NO: 1: Taq DNA polymerase amino acid sequence
SEQ ID NO: 2: Taq DNA polymerase nucleic acid sequence
SEQ ID NO: 3: Pyrococcus furiosus RFC-L PIP-box amino acid sequence
SEQ ID NO: 4: DNA polymerase variant Taq81 amino acid sequence
SEQ ID NO: 5: DNA polymerase variant Taq81 nucleic acid sequence
SEQ ID NO: 6: DNA polymerase variant Taq82 amino acid sequence
SEQ ID NO: 7: DNA polymerase variant Taq82 nucleic acid sequence
SEQ ID NO: 8: DNA polymerase variant Taq83 amino acid sequence
SEQ ID NO: 9: DNA polymerase variant Taq83 nucleic acid sequence
SEQ ID NO: 10: DNA polymerase variant Taq84 amino acid sequence
SEQ ID NO: 110: DNA polymerase variant Taq84 nucleic acid sequence
SEQ ID NO: 12: DNA polymerase variant Taq85 amino acid sequence
SEQ ID NO: 13: DNA polymerase variant Taq85 nucleic acid sequence
SEQ ID NO: 14: DNA polymerase variant Taq92 amino acid sequence
SEQ ID NO: 15: DNA polymerase variant Taq92 nucleic acid sequence
SEQ ID NO: 16: DNA polymerase variant Taq93 amino acid sequence
SEQ ID NO: 17: DNA polymerase variant Taq93 nucleic acid sequence
SEQ ID NO: 18: DNA polymerase variant Taq94 amino acid sequence
SEQ ID NO: 19: DNA polymerase variant Taq94 nucleic acid sequence
SEQ ID NO: 20: DNA polymerase variant Taq95 amino acid sequence
SEQ ID NO: 21: DNA polymerase variant Taq95 nucleic acid sequence
SEQ ID NO: 22: DNA polymerase variant Taq96 amino acid sequence
SEQ ID NO: 23: DNA polymerase variant Taq96 nucleic acid sequence
SEQ ID NO: 24: DNA polymerase variant Taq97 amino acid sequence
SEQ ID NO: 25: DNA polymerase variant Taq97 nucleic acid sequence
SEQ ID NO: 26: DNA polymerase variant Taq98 amino acid sequence
SEQ ID NO: 27: DNA polymerase variant Taq98 nucleic acid sequence
SEQ ID NO: 28: TaqNPIP-5 primer
SEQ ID NO: 29: Taq-3 primer
SEQ ID NO: 30: taqN10-5 primer
SEQ ID NO: 31: taqN10-3 primer
SEQ ID NO: 32: taqN15-5 primer
SEQ ID NO: 33: taqN15-3 primer
SEQ ID NO: 34: taq-plus20-5 primer
SEQ ID NO: 35: taq-plus20-3 primer
SEQ ID NO: 36: Taq-plus12-5 primer
SEQ ID NO: 37: Taq-plus12-3 primer
SEQ ID NO: 38: Taq-5 primer
SEQ ID NO: 39: Tq-L5-PIP-3 primer
SEQ ID NO: 40: Tq-L10-PIP-3 primer
SEQ ID NO: 41: Tq-L15-PIP-3 primer
SEQ ID NO: 42: F1 primer
SEQ ID NO: 43: R2-2 primer
SEQ ID NO: 44: LF-35 primer
SEQ ID NO: 45: LR8-35 primer
SEQ ID NO: 46: LR12-35 primer
SEQ ID NO: 47: LR15-35 primer
SEQ ID NO: 48: Taq95-5 primer
SEQ ID NO: 49: Taq96-5 primer
SEQ ID NO: 50: Taq97-5 primer
SEQ ID NO: 51: Taq98-5 primer
SEQ ID NO: 52: P. furiosus RFC-L PIP-box amino acid sequence
SEQ ID NO: 53: M. jannaschii RFC-L PIP-box amino acid sequence
SEQ ID NO: 54: P. furiosus PolBI PIP-box amino acid sequence
SEQ ID NO: 55: T. litoralis PolBI PIP-box amino acid sequence
SEQ ID NO: 56: A. fulgidus PolBI PIP-box amino acid sequence
SEQ ID NO: 57: P. occultum PolBII PIP-box amino acid sequence
SEQ ID NO: 58: M. jannaschii DP2 PIP-box amino acid sequence
SEQ ID NO: 59: M. thermoautotrophicum DP2 PIP-box amino acid sequence
SEQ ID NO: 60: H. sapiens Polδ p66 PIP-box amino acid sequence
SEQ ID NO: 61: S. cerevisiae Pol32 PIP-box amino acid sequence
SEQ ID NO: 62: S. pombe Cdc27 PIP-box amino acid sequence
SEQ ID NO: 63: S. cerevisiae Pol2 PIP-box amino acid sequence
SEQ ID NO: 64: P. furiosus Fen1 PIP-box amino acid sequence
SEQ ID NO: 65: M. jannaschii Fen1 PIP-box amino acid sequence
SEQ ID NO: 66: A. fulgidus Fen1 PIP-box amino acid sequence
SEQ ID NO: 67: H. sapiens Fen1 PIP-box amino acid sequence
SEQ ID NO: 68: D. melanogaster Fen1 PIP-box amino acid sequence
SEQ ID NO: 69: S. cerevisiae Fen1 PIP-box amino acid sequence
SEQ ID NO: 70: S. pombe Fen1 PIP-box amino acid sequence
SEQ ID NO: 71: H. sapiens DNA ligase I PIP-box amino acid sequence
SEQ ID NO: 72: X. laevis DNA ligase I PIP-box amino acid sequence
SEQ ID NO: 73: S. cerevisiae DNA ligase I PIP-box amino acid sequence
SEQ ID NO: 74: S. pombe DNA ligase I PIP-box amino acid sequence
SEQ ID NO: 75: H. sapiens MSH3 PIP-box amino acid sequence
SEQ ID NO: 76: S. cerevisiae MSH3 PIP-box amino acid sequence
SEQ ID NO: 77: H. sapiens MSH6 PIP-box amino acid sequence
SEQ ID NO: 78: S. cerevisiae MSH6 PIP-box amino acid sequence
SEQ ID NO: 79: H. sapiens UNG2 PIP-box amino acid sequence
SEQ ID NO: 80: S. cerevisiae UNG PIP-box amino acid sequence
SEQ ID NO: 81: H. sapiens hMYH PIP-box amino acid sequence
SEQ ID NO: 82: H. sapiens XPG PIP-box amino acid sequence
SEQ ID NO: 83: C. elegans XPG PIP-box amino acid sequence
SEQ ID NO: 84: S. cerevisiae XPG PIP-box amino acid sequence
SEQ ID NO: 85: S. pombe XPG PIP-box amino acid sequence
SEQ ID NO: 86: S. cerevisiae Cac1 PIP-box amino acid sequence
SEQ ID NO: 87: H. sapiens hRECQ5 PIP-box amino acid sequence
SEQ ID NO: 88: S. cerevisiae Rrm3 PIP-box amino acid sequence
SEQ ID NO: 89: H. sapiens Cdc25C PIP-box amino acid sequence
SEQ ID NO: 90: H. sapiens p15 PIP-box amino acid sequence
SEQ ID NO: 91: H. sapiens DNA-dependent protein kinase PIP-box amino acid sequence

1. A fusion polypeptide comprising, in a direction of from an N-terminal side to a C-terminal side,
a) one or more peptides which bind to a PCNA, and
b) a polypeptide having a DNA polymerase activity.

2. The fusion polypeptide according to claim 1, characterized in that the peptide which bind to a PCNA is a peptide comprising a PIP box.

3. The fusion polypeptide according to claim 2, characterized in that the PIP box is a peptide consisting of an amino acid sequence shown in any one of SEQ ID NOs: 52 to 91 of the Sequence Listing.

4. The fusion polypeptide according to claim 1 or 2, characterized in that the peptide which bind to a PCNA is a peptide comprising a PIP box derived from a DNA polymerase-associated factor.

5. The fusion polypeptide according to claim 1, characterized in that the peptide which bind to a PCNA is a peptide comprising a PIP box derived from a replication factor C large subunit.

6. The fusion polypeptide according to claim 1, characterized in that the peptide which bind to a PCNA is the amino acid sequence shown in SEQ ID NO: 3 of the Sequence Listing, or a peptide comprising the above amino acid sequence.

7. The fusion polypeptide according to claim 1, characterized in that the fusion polypeptide comprises a 5 to 50 amino acid linker peptide between the peptide which bind to a PCNA and the polypeptide having a DNA polymerase activity.

8. The fusion polypeptide according to claim 7, characterized in that the linker peptide is an amino acid sequence composed of serine and glycine.

9. The fusion polypeptide according to claim 1, characterized in that the polypeptide having a DNA polymerase activity is a Pol I-type DNA polymerase or a fragment thereof.

10. The fusion polypeptide according to claim 1, characterized in that the polypeptide having a DNA polymerase activity is a Taq DNA polymerase or a fragment thereof.

11. A nucleic acid encoding a fusion polypeptide as defined in claim 1.

12. A composition for amplifying nucleic acids comprising a fusion polypeptide as defined in claim 1.

13. The composition for amplifying nucleic acids according to claim 12, further containing a PCNA.

14. A kit comprising a fusion polypeptide as defined in claim 1.

15. The kit according to claim 14, further comprising a PCNA.

16. A method for producing a DNA complementary to a template DNA, characterized by the use of a composition comprising a fusion polypeptide as defined in claim 1 and a PCNA.


SEQUENCE LISTING (91 sequences)

SEQ ID NO: 1 - protein, 832 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - DNA, 2499 bp, Thermus aquaticus
atgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 3 - protein, 11 aa, Pyrococcus furiosus
KQATLFDFLKK

SEQ ID NO: 4 - protein, 851 aa, Artificial Sequence
  DNA polymerase variant Taq81
MSGKQATLFDFLKKGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - DNA, 2556 bp, Artificial Sequence
  DNA polymerase variant Taq81
atgagcggcaagcaggcgacgctgttcgacttcctcaagaagggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 6 - protein, 856 aa, Artificial Sequence
  DNA polymerase variant Taq82
MSGKQATLFDFLKKSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - DNA, 2571 bp, Artificial Sequence
  DNA polymerase variant Taq82
atgagcggcaagcaggcgacgctgttcgacttcctcaagaagagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 8 - protein, 861 aa, Artificial Sequence
  DNA polymerase variant Taq83
MSGKQATLFDFLKKGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 9 - DNA, 2586 bp, Artificial Sequence
  DNA polymerase variant Taq83
atgagcggcaagcaggcgacgctgttcgacttcctcaagaagggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 10 - protein, 881 aa, Artificial Sequence
  DNA polymerase variant Taq84
MSGKQATLFDFLKKGSGSGSGSGSGSGSGSGSGSGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 11 - DNA, 2646 bp, Artificial Sequence
  DNA polymerase variant Taq84
atgagcggcaagcaggcgacgctgttcgacttcctcaagaagggttctggttccggctccggcagcggctctggctccggctccggtagcggttccggtagcggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 12 - protein, 893 aa, Artificial Sequence
  DNA polymerase variant Taq85
MSGKQATLFDFLKKGSGSGSGSGSGSGSGSGSGSGSGSGSGSGSGSGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 13 - DNA, 2682 bp, Artificial Sequence
  DNA polymerase variant Taq85
atgagcggcaagcaggcgacgctgttcgacttcctcaagaagggttctggttccggctccggcagcggctctggctccggctccggtagcggttccggtagcggctctggtagcggctctggtagcggttccggtagcggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 14 - protein, 848 aa, Artificial Sequence
  DNA polymerase variant Taq92
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKESGSGSKQATLFDFLKK

SEQ ID NO: 15 - DNA, 2547 bp, Artificial Sequence
  DNA polymerase variant Taq92
atgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagagcggttctggctccaagcaggcgacgctgttcgacttcctcaagaagtga

SEQ ID NO: 16 - protein, 853 aa, Artificial Sequence
  DNA polymerase variant Taq93
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKESGSGSSGSGSKQATLFDFLKK

SEQ ID NO: 17 - DNA, 2562 bp, Artificial Sequence
  DNA polymerase variant Taq93
atgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagagcggttctggctcctctggcagcggttccaagcaggcgacgctgttcgacttcctcaagaagtga

SEQ ID NO: 18 - protein, 858 aa, Artificial Sequence
  DNA polymerase variant Taq94
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKESGSGSSGSGSGSGSGKQATLFDFLKK

SEQ ID NO: 19 - DNA, 2577 bp, Artificial Sequence
  DNA polymerase variant Taq94
atgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagagcggttctggctcctctggcagcggttccggtagcggctccggcaagcaggcgacgctgttcgacttcctcaagaagtga

SEQ ID NO: 20 - protein, 885 aa, Artificial Sequence
  DNA polymerase variant Taq95
MSGKQATLFDFLKSGSGSGSGSGSGSGKQATLFDFLKKGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 21 - DNA, 2658 bp, Artificial Sequence
  DNA polymerase variant Taq95
atgtccggtaaacaagccacccttttcgattttctgaaaagcggttctggttctggcagcggtagcggctccggcagcggcaagcaggcgacgctgttcgacttcctcaagaagggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 22 - protein, 909 aa, Artificial Sequence
  DNA polymerase variant Taq96
MSGKQATLFDFLKSGSGSGSGSGSGSGKQATLFDFLKSGSGSGSGSGSGSGKQATLFDFLKKGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 23 - DNA, 2730 bp, Artificial Sequence
  DNA polymerase variant Taq96
atgagcggcaaacaggctacactctttgactttctgaaaagcggcagcggttccggctctggtagcggctctggttccggtaaacaagccacccttttcgattttctgaaaagcggttctggttctggcagcggtagcggctccggcagcggcaagcaggcgacgctgttcgacttcctcaagaagggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 24 - protein, 933 aa, Artificial sequence
  DNA polymerase variant Taq97
MSGKQATLFDFLKSGSGSGSGSGSGSGKQATLFDFLKSGSGSGSGSGSGSGKQATLFDFLKSGSGSGSGSGSGSGKQATLFDFLKKGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 25 - DNA, 2802 bp, Artificial sequence
  DNA polymerase variant Taq97
atgagcggtaaacaagcaactcttttcgatttccttaagtctgggagtgggagcggttccggcagcggttcgggcagcggcaaacaggctacactctttgactttctgaaaagcggcagcggttccggctctggtagcggctctggttccggtaaacaagccacccttttcgattttctgaaaagcggttctggttctggcagcggtagcggctccggcagcggcaagcaggcgacgctgttcgacttcctcaagaagggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 26 - protein, 957 aa, Artificial sequence
  DNA polymerase variant Taq98
MSAKQATLFDFLKSGSGSGSGSGSGSGKQATLFDFLKSGSGSGSGSGSGSGKQATLFDFLKSGSGSGSGSGSGSGKQATLFDFLKSGSGSGSGSGSGSGKQATLFDFLKKGSGSGSGSGSGSGSGMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 27 - DNA, 2874 bp, Artificial sequence
  DNA polymerase variant Taq98
atgtccgctaagcaagccacgctgttcgactttctcaaatccggctcgggcagtgggagcggctctggcagcgggagcggtaaacaagcaactcttttcgatttccttaagtctgggagtgggagcggttccggcagcggttcgggcagcggcaaacaggctacactctttgactttctgaaaagcggcagcggttccggctctggtagcggctctggttccggtaaacaagccacccttttcgattttctgaaaagcggttctggttctggcagcggtagcggctccggcagcggcaagcaggcgacgctgttcgacttcctcaagaagggctctggtagcggtagcggttctggctccggtagcggctccggcatgcgtggtatgctgccgctgttcgaacccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 28 - DNA, 85 bp, Artificial Sequence
  TaqNPIP-5 primer
cgcgcatatgagcggcaagcaggcgacgctgttcgacttcctcaagaagggtagcggctccggcatgcgtggtatgctgccgctg

SEQ ID NO: 29 - DNA, 37 bp, Artificial Sequence
  Taq-3 primer
gcgcgcggccgctcactccttggcggagagccagtcc

SEQ ID NO: 30 - DNA, 52 bp, Artificial Sequence
  taqN10-5 primer
gctgttcgacttcctcaagaagagcggttctggctccggtagcggctccggc

SEQ ID NO: 31 - DNA, 52 bp, Artificial Sequence
  taqN10-3 primer
gccggagccgctaccggagccagaaccgctcttcttgaggaagtcgaacagc

SEQ ID NO: 32 - DNA, 67 bp, Artificial Sequence
  taqN15-5 primer
gctgttcgacttcctcaagaagggctctggtagcggtagcggttctggctccggtagcggctccggc

SEQ ID NO: 33 - DNA, 67 bp, Artificial Sequence
  taqN15-3 primer
gccggagccgctaccggagccagaaccgctaccgctaccagagcccttcttgaggaagtcgaacagc

SEQ ID NO: 34 - DNA, 100 bp, Artificial Sequence
  taq-plus20-5 primer
gctgttcgacttcctcaagaagggttctggttccggctccggcagcggctctggctccggctccggtagcggttccggtagcggctctggtagcggtagc

SEQ ID NO: 35 - DNA, 100 bp, Artificial Sequence
  taq-plus20-3 primer
gctaccgctaccagagccgctaccggaaccgctaccggagccggagccagagccgctgccggagccggaaccagaacccttcttgaggaagtcgaacagc

SEQ ID NO: 36 - DNA, 72 bp, Artificial Sequence
  Taq-plus12-5 primer
ggctctggctccggctccggtagcggttccggtagcggctctggtagcggctctggtagcggttccggtagc

SEQ ID NO: 37 - DNA, 72 bp, Artificial Sequence
  Taq-plus12-3 primer
gctaccggaaccgctaccagagccgctaccagagccgctaccggaaccgctaccggagccggagccagagcc

SEQ ID NO: 38 - DNA, 28 bp, Artificial Sequence
  Taq-5 primer
gcgccccatatgcgtggtatgctgccgc

SEQ ID NO: 39 - DNA, 77 bp, Artificial Sequence
  Tq-L5-PIP-3 primer
ggggcggccgctcacttcttgaggaagtcgaacagcgtcgcctgcttggagccagaaccgctctccttggcggagag

SEQ ID NO: 40 - DNA, 92 bp, Artificial Sequence
  Tq-L10-PIP-3 primer
ggggcggccgctcacttcttgaggaagtcgaacagcgtcgcctgcttggaaccgctgccagaggagccagaaccgctctccttggcggagag

SEQ ID NO: 41 - DNA, 107 bp, Artificial Sequence
  Tq-L15-PIP-3 primer
ggggcggccgctcacttcttgaggaagtcgaacagcgtcgcctgcttgccggagccgctaccggaaccgctgccagaggagccagaaccgctctccttggcggagag

SEQ ID NO: 42 - DNA, 35 bp, Artificial Sequence
  F1 primer
gagttcgtgtccgtacaactggcgtaatcatggcc

SEQ ID NO: 43 - DNA, 27 bp, Artificial Sequence
  R2-2 primer
cttttcagcctggccctttcctttacc

SEQ ID NO: 44 - DNA, 35 bp, Artificial Sequence
  LF-35 primer
tgctgaaatgaattctaagcggagatcgcctagtg

SEQ ID NO: 45 - DNA, 35 bp, Artificial Sequence
  LR8-35 primer
atgggcaatacgaacgacggcaatgattgccagag

SEQ ID NO: 46 - DNA, 35 bp, Artificial Sequence
  LR12-35 primer
atatacgccgagatctttagctgtcttggtttgcc

SEQ ID NO: 47 - DNA, 35 bp, Artificial Sequence
  LR15-35 primer
ctcgctttccactccagagccagtctcgcttcgtc

SEQ ID NO: 48 - DNA, 100 bp, Artificial Sequence
  Taq95-5 primer
gcgccatatgtccggtaaacaagccacccttttcgattttctgaaaagcggttctggttctggcagcggtagcggctccggcagcggcaagcaggcgacg

SEQ ID NO: 49 - DNA, 100 bp, Artificial Sequence
  Taq96-5 primer
gcgccatatgagcggcaaacaggctacactctttgactttctgaaaagcggcagcggttccggctctggtagcggctctggttccggtaaacaagccacc

SEQ ID NO: 50 - DNA, 100 bp, Artificial Sequence
  Taq97-5 primer
gcgccatatgagcggtaaacaagcaactcttttcgatttccttaagtctgggagtgggagcggttccggcagcggttcgggcagcggcaaacaggctaca

SEQ ID NO: 51 - DNA, 100 bp, Artificial Sequence
  Taq98-5 primer
gcgccatatgtccgctaagcaagccacgctgttcgactttctcaaatccggctcgggcagtgggagcggctctggcagcgggagcggtaaacaagcaact

SEQ ID NO: 52 - protein, 8 aa, Pyrococcus furiosus
QATLFDFL

SEQ ID NO: 53 - protein, 8 aa, Methanococcus jannaschii
QLTLDAFF

SEQ ID NO: 54 - protein, 8 aa, Pyrococcus furiosus
QVGLTSWL

SEQ ID NO: 55 - protein, 8 aa, Thermococcus litoralis
QTGLDAWL

SEQ ID NO: 56 - protein, 8 aa, Archaeoglobus fulgidus
QMSLDSFF

SEQ ID NO: 57 - protein, 8 aa, Pyrodictium occultum
QRSLFDFF

SEQ ID NO: 58 - protein, 8 aa, Methanococcus jannaschii
QVKLSDFF

SEQ ID NO: 59 - protein, 8 aa, Methanobacterium thermoautotrophicum
QSSLDVFL

SEQ ID NO: 60 - protein, 8 aa, Homo sapiens
QVSITGFF

SEQ ID NO: 61 - protein, 8 aa, Saccharomyces cerevisiae
QGTLESFF

SEQ ID NO: 62 - protein, 8 aa, Schizosaccharomyces pombe
QKSIMSFF

SEQ ID NO: 63 - protein, 8 aa, Saccharomyces cerevisiae
QTSLTKFF

SEQ ID NO: 64 - protein, 8 aa, Pyrococcus furiosus
QSTLESWF

SEQ ID NO: 65 - protein, 8 aa, Methanococcus jannaschii
QKTLDAWF

SEQ ID NO: 66 - protein, 8 aa, Archaeoglobus fulgidus
QATLERWF

SEQ ID NO: 67 - protein, 8 aa, Homo sapiens
QGRLDDFF

SEQ ID NO: 68 - protein, 8 aa, Drosophila melanogaster
QVRLDSFF

SEQ ID NO: 69 - protein, 8 aa, Saccharomyces cerevisiae
QGRLDGFF

SEQ ID NO: 70 - protein, 8 aa, Schizosaccharomyces pombe
QGRLDSFF

SEQ ID NO: 71 - protein, 8 aa, Homo sapiens
QRSIMSFF

SEQ ID NO: 72 - protein, 8 aa, Xenopus laevis
QRTIKSFF

SEQ ID NO: 73 - protein, 8 aa, Saccharomyces cerevisiae
QATLARFF

SEQ ID NO: 74 - protein, 8 aa, Schizosaccharomyces pombe
QSDISNFF

SEQ ID NO: 75 - protein, 8 aa, Homo sapiens
QAVLSRFF

SEQ ID NO: 76 - protein, 8 aa, Saccharomyces cerevisiae
QPTISRFF

SEQ ID NO: 77 - protein, 8 aa, Homo sapiens
QSTLYSFF

SEQ ID NO: 78 - protein, 8 aa, Saccharomyces cerevisiae
QSSLLSFF

SEQ ID NO: 79 - protein, 8 aa, Homo sapiens
QKTLYSFF

SEQ ID NO: 80 - protein, 8 aa, Saccharomyces cerevisiae
QTTIEDFF

SEQ ID NO: 81 - protein, 8 aa, Homo sapiens
QQVLDNFF

SEQ ID NO: 82 - protein, 8 aa, Homo sapiens
QLRIDSFF

SEQ ID NO: 83 - protein, 8 aa, Caenorhabditis elegans
QMRLDRFF

SEQ ID NO: 84 - protein, 8 aa, Saccharomyces cerevisiae
QKRINEFF

SEQ ID NO: 85 - protein, 8 aa, Schizosaccharomyces pombe
QSNLTQFF

SEQ ID NO: 86 - protein, 8 aa, Saccharomyces cerevisiae
QSRIGNFF

SEQ ID NO: 87 - protein, 8 aa, Homo sapiens
QNLIRHFF

SEQ ID NO: 88 - protein, 8 aa, Saccharomyces cerevisiae
QQTLSSFF

SEQ ID NO: 89 - protein, 8 aa, Homo sapiens
QEELFNFF

SEQ ID NO: 90 - protein, 8 aa, Homo sapiens
QKGIGEFF

SEQ ID NO: 91 - protein, 8 aa, Homo sapiens
QLIIRNFW